(19) United States
(12) Patent Application Publication
(10) Pub. No.: US 2015/0147382 A1
(43) Pub. Date: May 28, 2015

(54) TOPICAL LIPOSOMAL COMPOSITIONS FOR DELIVERING HYDROPHOBIC DRUGS AND METHODS PREPARING SAME

(71) Applicant: Exir Nano Sina Company, Tehran (IR)

(72) Inventors: Mahmoud Reza Jaafari, Mashhad (IR); Ali Khamesipour, Tehran (IR)

(73) Assignee: Exir Nano Sina Company, Tehran (IR)

(21) Appl. No.: 14/491,724

(22) Filed: Sep. 19, 2014

Related U.S. Application Data
(60) Provisional application No. 61/880,984, filed on Sep. 23, 2013.

(51) Int. Cl. A61K 31/7048; A61K 9/00; A61K 9/127

(52) U.S. Cl. CPC A61K 31/7048; A61K 9/127; A61K 9/1277; A61K 9/0014
USPC 424/450; 514/31

(57) ABSTRACT

A topical liposomal composition of Amphotricin B (AmB) and method for preparing same, which can be used as a composition and method for preparing topical liposomal compositions of other hydrophobic drugs. The formulation using AmphotricinB can be used for treating fungal or protozoan infections. The composition is stable with no significant changes in the sizes and AmB content of liposomes after storing at 4° C. and room temperature (22° C.) for more than 20 months. In in vivo and in vitro testing, the compositions exhibit high efficacy in treating cutaneous leishmaniasis.

### CROSS REFERENCE TO RELATED APPLICATION

The present invention claims priority from pending U.S. Provisional Patent Application Ser. No. 61/880,984, filed Sep. 23, 2013, entitled “Topical Liposomal Composition for Delivering Hydrophobic Drug,” the subject matter of which is incorporated by reference herein in its entirety.

### SPONSORSHIP STATEMENT

This application has been sponsored by the Iranian Nanotechnology Initiative Council, which does not have any rights in this application.

### TECHNICAL FIELD

The present invention relates, in general, to a hydrophobic drug delivery composition and method of preparing same, and to a liposomal composition for topical use and method preparing the composition.

### BACKGROUND OF THE INVENTION

As is known in the art, liposomes are colloidal particles, typically consisting of phospholipid and cholesterol. These lipid molecules form bilayers, which entrap water-soluble molecules in their internal water compartment and water-insoluble ones into their lipid bilayers. Liposomes, in proper formulations and sizes, deliver drugs to the skin based on the similarity of the bilayers structure of lipid vesicles to that of natural membranes and target the macrophages within the dermis. Liposomes have been shown to be able to pass through the strateum corneum (SC), and reach the epidermis and deep dermis, as well as target the macrophages within the dermis. The presence of intact liposomes has also been shown in the epidermis and dermis by electron microscopy studies.

Liposomes, in proper formulations and sizes, thus can deliver drugs to the skin based on the similarity of the bilayers structure of lipid vesicles to that of natural membrane and target the macrophages within the dermis. It is speculated that after topical application of liposomes, at least some of the vesicles, especially those with sizes around 100 nm and less, pass through the SC of intact skin and reach the epidermis and deep dermis layers.

Amphotericin B (AmB), a known hydrophobic drug, is a polyene produced from the natural fermentation of Streptomyces nodosus, which indicates effectiveness for the treatment of fungal and protozoan infections, and also for visceral leishmaniasis. Visceral leishmaniasis (VL), also known as kala-azar, black fever, and Dumdum fever, is the most severe form of Leishmaniasis. However, the major drawbacks arising from the amphotericin B in the development of pharmaceutical formulations are their low aqueous solubility and amphotericin B's poor physical and chemical stability. For this reason, parallel research has been conducted to launch new antifungal drugs in this increasingly sophisticated area, and there have been various studies to try to increase the solubility of amphotericin B in an aqueous media, as well as improve its stability.

Regarding these drawbacks, as well as potential toxic adverse effects of AmB, three different lipid-based formulations of AmB, Amphotec™, Abelcet™ and AmBisome,™ have been developed and commercialized in the United States and Europe. Of these formulations, AmBisome™ has significantly lower toxicity compared to the other formulations. However, in most cases, the lipid base formulation of AmB is administered by the intravenous route. With regard to AmB, since there is considerable cost and minimum impact via injection, topical use is considered as a preferred drug delivery method due to need for lower doses, as well the low possibility of the drug reaching to sensitive internal organs, such as the kidneys.

However, the formidable barrier nature of the stratum corneum (SC) of skin does not allow the penetration of drugs like as AmB. The Leishmania parasite, however, lives and multiplies within the phagolysosome of macrophages in the deep dermal layer of skin. Thus, topically-applied drugs for the treatment of cutaneous leishmaniasis (CL) must be able to target the Leishmania parasites within the phagolysosome of the infected macrophages in the deep dermal layers of the skin. To get the infected macrophages in the dermis, one can use Liposomal AmphotricinB (AmB) in an encapsulated drug, released in the phagolysosome of the macrophage by acidic lysosomal enzymes where Leishmania parasite lives and multiply. Thus, AmB in this way targets the phagolysosome of the macrophage, and since the AmB would be in direct contact of fungal or protozoal infections or Leishmania parasite, then it can kill the parasites very efficiently.

A conventional topical preparation of AmB in soft white paraffin containing 12% methylbenzethonium chloride, however, has not been effective in the treatment of cutaneous L. major lesions in BALB/c mice. In order to obtain an effective drug penetration through the skin, and target parasites like the Leishmania parasites within the phagolysosome of the infected macrophages in the deep dermal layer of skin using AmB, an efficacious drug-carrier system like liposomes might be helpful.

There is, therefore, a present need for such efficacious drug-carrier systems using liposomes for delivery.

These and many other objects are met in various embodiments of the present invention, offering significant advantages over the known prior art.

### SUMMARY OF THE INVENTION

In present invention, liposomal AmB compositions and methods of making them are disclosed where the methods and compositions can also be used for other hydrophobic drugs. Preferred liposome diameters are around 100 nm since there are no significant changes in the sizes and AmB content of liposomes when stored at 4° C. and room temperature (22° C.) for more than 20 months.

In one embodiment of the present invention, the liposomal Amphotricin B formulation is for topical use. For example, liposomal Amphotricin B compositions can be used for treating antifungal and antiprotozoal infections, and additionally treat cutaneous leishmaniasis, where Liposomes containing 0.1, 0.2, 0.4% AmB (Lip-AmB) were prepared by using Soya PC, cholesterol, solvent system and some additives. Phospholipon 90G.

In another embodiment of the present invention, the said formulation and method for preparing same can be used for topically-delivering other hydrophobic drugs, including cytotoxic drugs like docetaxel, paclitaxel and SN38; hormones like tamoxifen; immunosuppressive drugs like cyclosporine A; natural compounds like curcumin and resveratrol; corticosteroids like triamicinolone; antifungal and antileishmanial drugs like amphotricin B, miltofesine and nystatin, as well as combinations thereof.

In still another embodiment of the present invention, solvent components, which are used for dissolving Amb, include DMSO and the other solvents used for dissolving lipid components.

In yet another embodiment of the present invention, a method is disclosed for preparing topical liposomal Amb, which involves dissolving lipid and AmB in proper solvents, and further activities to acquire the final composition having nanosize liposomes are disclosed.

In a further embodiment of the present invention, the characterization of prepared liposomes is analyzed, which shows liposome diameters of around 100 nanometers.

In another embodiment of the present invention, the stability of the prepared liposomal AmB is analyzed, where it showed high stability with no significant changes in the sizes and AmB content of liposomes when they are stored at 4° C. and room temperature (22° C.) for more than 20 months. In one aspect, the minimum inhibitory concentration (MIC) of Lip-AmB formulations and Fungizone™ against L. major promastigotes in a culture was 0.625 and 0.5 μg/ml, respectively, and remained unchanged when stored at 4° C. and room temperature for more than 20 months.

In yet another embodiment of the present invention, the penetration of the prepared AmB from the liposomal AmB composition through and into skin was evaluated by in vitro, using Franz diffusion cells fitted with mice skin at 37° C. for 8 hours, where the in vitro permeation data showed that almost 4% of the applied Lip-AmB composition penetrated across mice skin and the retained amount in the skin was around 60%.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying DRAWINGS, where like reference numerals designate like structural and other elements, in which:

FIG. 1 is a chart illustration of the effect of topical liposomal AmB on the course of a disease in a BALB/c mice L. major model of cutaneous leishmaniasis;

FIGS. 2A and 2B are charts illustrating the splenic parasite burden in BALB/c mice treated with topical liposomal AmB pursuant to the principles of the present invention; and

FIGS. 3A and 3B are charts illustrating the infected lesion parasite burden in BALB/c mice treated with topical liposomal AmB pursuant to the teachings of the present invention.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

The lipid components, namely phospholipid and cholesterol, along with other additives, are dissolved in a sufficient amount of a solvent preferably containing propylene glycol and glycerol in a concentration of about 5 to about 15% w/w. The lipid components used preferably include phospholipids at concentrations of about 5 to about 20% w/w, and cholesterol at concentrations of about 0.5 to about 5% w/w. The lipid components of the formulations could be dissolved in these solvents at 65-75° C., which takes a few hours depending on batch size. As is understood in the art, a suitable mixer depending on the batch size is needed to dissolve the lipids in the solvents. The additives used in this step preferably include at least one antioxidant, penetration enhancer and antimicrobial preservative. As an antioxidant, vitamin E and butylated hydroxyl anisole (BHT) could be used at concentrations of about 0.1 to about 0.5% w/w. Oleic acid (OA) could be used in the formulation as a penetration enhancer at concentrations from about 0.5 to about 5% w/w. Methyl parabene (MP, 0.05-0.5% w/w), propyl parabene (PP, 0.001-0.02% w/w) and phenol (0.2-0.5% w/w) could be used in the formulations as an antimicrobial preservative.

To dissolve the drug (AmB), a solvent containing Dimethylsulfoxide (DMSO) is preferred. Depending on the concentration of AmB, DMSO is used at about 1-10% w/w concentration in the formulations. Dissolving the drug requires about one hour at about 60-75° C. As is understood, a suitable mixer, depending on the batch size, could be needed to dissolve the AmB in the solvent.

After complete dissolving of the lipid components, the dissolved drug in the solvent is added and the mixture is heated at a temperature of about 60-75° C. to obtain a uniform lipid phase.

An aqueous phase is prepared by using sterile distilled water or a suitable buffer like succinate, histidine, citrate or phosphates at pH ranges from 5.5 to 7.5. To stabilize the formulation, an emulsifier, such as Triethanlamine (TEA) in concentration ranges from about 0.5 to about 2% w/w in this step can be used, which is dissolved in the aqueous phase of the formulations. The aqueous phase containing TEA (1%) and disodium succinate hexahydrate buffer (10 mM; pH: 5.5) up to 100% was separately heated to about 75° C. and then added to the lipid phase and mixed. Finally, the mixture was homogenized using an Ultra-Turrax IKA T10 homogenizer (IKA Werke GmbH & Co. KG, Staufen, Germany) for 10 min at 5,000 rpm to obtain the final product, which contains liposomes with diameters of about 100 nm.

### Materials: Chemicals

Amphotericin B USP 32 was purchased from ASENCE Pharma Private Limited (Vadodara, India). Soya PC was purchased from Lipoid GmbH (Ludwigshafen, Germany). Cholesterol (chol), propylene glycol (PG), methyl parabene (MP), propyl parabene (PP), triethanlamine (TEA), dimethyl sulfoxide (DMSO), disodium succinate hexahydrate, oleic acid (OA) and vitamin E were purchased from Merck (Darmstadt, Germany). Alamar Blue was purchased from Biosource (International, Inc., USA). All other chemicals were of reagent grade and were used as received.

### Animals and Parasites

Female BALB/c mice, 6-8 weeks old, were purchased from the Pasteur Institute in Tehran, Iran. The mice were maintained in the Animal House of Nanotechnology Research Center of Mashhad University of Medical Sciences, and fed with tap water and a standard laboratory diet. The animals were housed in a colony room at a 12/12 hours light/dark cycle at 21° C., and had free access to water and food. Animal experiments were carried out according to the standards of the Mashhad University of Medical Sciences, Ethical Committee Acts.

The virulence of a Leishmania major strain (MRHO/IR/75/ER) was maintained with passage in BALB/c mice. The amastigotes were isolated from the spleens of infected mice, and cultured in an NNN medium and subcultured in RPMI 1640 (Sigma), containing 10% v/v heat-inactivated FCS, 2 mM glutamine, 100 U/ml of penicillin and 100 μg/ml of streptomycin sulfate (RPMI-FCS) at 25±1° C.

### Example 1

### A Topical Liposomal AmB

The lipid components employed in practicing the present invention preferably contain Soya PC (10-20% w/w), Chol (1-5% w/w), PG (5-10% w/w), vitamin E (0.1-0.5% w/w), MP (0.06-0.2% w/w), PP (0.001-0.02% w/w) and OA (2% w/w), which were melted at temperatures of around 70° C. (lipid melt). After the complete dissolving of the aforesaid lipids, a dissolved drug containing AmB (0.2% w/w), which had been dissolved in DMSO (5% w/w) and heated at 70° C. (AmB dissolved in DMSO at concentration of 35 mg/ml), is added, and the mixture then heated at about 70° C. Along with this step, an aqueous phase containing TEA (1% w/w) dissolved in water and diluted up to 100% w/w was separately heated at a temperature around 70° C. and then added to the lipid phase, and mixed with a suitable mixer at about 70° C. for 15 minutes. Finally, the mixture was homogenized preferably using Ultra-Turrax IKA T10 homogenizer (IKA Werke GmbH & Co. KG, Staufen, Germany) for 10 min at 5,000 rpm to obtain the final product.

### Example 2

### Characterization of Liposomes

The physical appearance of formulations produced using the techniques set forth in the present invention was yellow paste like. The prepared liposomes had average diameters of around 100 nm, as shown in connection with TABLE 1 hereinbelow. As indicated, there were no significant differences in the size of control liposomes and Lip-AmB at different concentrations.

The concentration of AmB in liposomal formulations was determined by a spectrophotometeric procedure. With reference to TABLE 2, the table illustrates the AmB concentrations calculated from the aforesaid spectrophotometric procedure, and the results for different formulations concerning the prepared formulations.

The particle diameter of each sample was measured in triplicate by dynamic light scattering. As noted in TABLE 1, particle size distribution of liposomal AmB formulations is observed.

TABLE 1
Particle size distribution of liposomal AmB formulations

| | Liposome Diameter |
|---|---|
| Formulation | (nm) ± SD |
| Control empty liposomes | 106.3 ± 2.1 |
| Lip-AmB 0.1% | 119.2 ± 14.4 |
| Lip-AmB 0.2% | 116.7 ± 9.9 |
| Lip-AmB 0.4% | 113.5 ± 10.4 |

n = 3, Mean ± SD

### Drug Analysis for Liposomal AmB Formulations

The amphotericin B concentration of the final formulation was measured by diluting the sample 1/1000 in methanol, measuring the absorbance at 406 nm, and comparing the absorption to a standard curve prepared from solid amphotericin B diluted in methanol. The standard curve was linear up to 6 micrograms amphotericin B per mL methanol. The intra- and inter-day variation for AmB was performed, and there was no significant difference between the day-to-day analyses. The validation results were repeated three times per concentration and at 5 concentrations.

With reference now to TABLE 2 hereinbelow, there is shown in detail the AmB concentrations calculated from the spectrophotometer results for liposome AmB formulations at different concentrations.

TABLE 2
The AmB concentrations for liposome AmB
formulations at different concentrations

| | AmB |
|---|---|
| | Concentration |
| Formulation | (mg/g) ± SD |
| Lip-AmB 0.1% | 1.17 ± 0.0058 |
| Lip-AmB 0.2% | 2.36 ± 0.0058 |
| Lip-AmB 0.4% | 4.11 ± 1.53 |

n = 3, Mean ± SD

### Example 3

### Cell Diffusion Study

In this study, jacketed Franz cells with a receiver volume of 40 ml were used and every experiment was conducted in triplicate at 37° C. Phosphate buffers of pH 7.4 were used as the receiver medium. A suitable size of full-thickness skin of BALB/c mice was cut and mounted in the Franz cell, with the stratum corneum side facing upward. The mice were shaved properly using an electrical clipper a day before the experiment. The membranes were initially left in the Franz cells for 30 min in order to facilitate hydration. Subsequently, 0.25 g of liposomal AmB was deposited onto each membrane surface.

A 250 μl aliquot of receiver solution was withdrawn from each receiver solution at one-hour intervals and replaced with the same volume of blank phosphate buffered saline (PBS) solution. Aliquots of the collected samples were analyzed for AmB content as explained hereinabove. The derived concentration values were corrected using the equation:

Mt(n)=Vr×Cn+Vs×ΣCm

where Mt(n) is the current cumulative mass of drug transport across the skin at time t, Cn the current concentration in the receiver medium, ΣCm the summed total of the previous measured concentrations, Vr the volume of the receiver medium, and the Vs correspond to the volume of the sample removed for analysis.

### Example 4

### In Vitro Promastigote Assay to Determine Liposomal AmB MICs

A microtiter dilution assay was used to determine minimum inhibitory concentrations (MICs) of AmB in different Lip-AmB formulations for the L. major promastigotes. A series of twofold dilutions of each AmB formulation (0.31 to 10 μg/ml) in RPMI-MOPS were prepared, and 100-μl aliquots of each drug dilution were dispensed into triplicate wells of a 96-well flat-bottom microtiter plate. Final AmB concentrations in the wells ranged from 0.156 to 5 μg/ml. Parasites were harvested at stationary phase of culture and aliquots (2.5×10⁶ promastigotes/100 μl/well) of L. major promastigotes were then dispensed into the appropriate wells. Alamar blue (20 μl/well) was added to all wells, and the plate was incubated at 25° C. for 48 h. Negative control wells contained 100 μl of RPMI-MOPS and 100 μl of the drug at 10 μg/ml; positive control wells were made up of 100 μl of RPMI-MOPS and 100 μl of the L. major promastigotes. The MIC was defined as the lowest concentration of the drug preventing the development of a red color.

### Example 5

### In Vitro Amastogote Assay

In this analysis, cells of the J774 A.1 mouse macrophage cell line were dispensed at a concentration of 50,000 macrophages/well into eight-well Lab-Tek (Nunc) chamber slides, and maintained at 37° C. in 5% CO2 for 24 hours to allow attachment of the cells. The cells were then infected with L. major promastigotes at a ratio of five promastigotes per macrophage, and incubated at 37° C. in 5% CO₂ for 24 hours to allow internalization of the parasites in the cells. The excess amount of promastigotes was removed by gently washing the cells with PBS three times, and the infected cells were incubated for an additional 24 hours to allow the establishment of the infection. The cells were then exposed to different concentrations of liposomal AmB formulations in triplicate for 2 days. The experiment was terminated by methanol fixation of the slides. The slides were then stained with Giemsa and evaluated microscopically to calculate the percentage of infected cells. The ED50 for each formulation was calculated by the CalcuSyn software Version 2.1 (Biosoft, Cambridge, UK).

### Example 6

### Stability Studies

In stability studies of liposomal AmB formulations, the liposomal AmB preparations were kept in 4° C. and room temperature (22° C.) and analyzed periodically for their particle diameters, AmB concentration, and biological activity of the AmB as determined by a promastigote assay.

In in vivo experiments, female 6-8 weeks BALB/c mice were inoculated subcutaneously at the base of the tail with 2×10⁶ L. major promastigotes harvested at stationary-phase. At 4 weeks post-infection, lesions were measured with calipers in two dimensions, mean diameters determined and mice were randomly divided into groups of 10. No significant differences (P>0.05) were seen in lesion size among the different groups. The lesions were then treated topically by 50 mg formulations twice a day for 4 weeks. The lesions sizes were measured weekly during treatment and at week 4 after the treatment was stopped.

With regard to quantitative parasite burden, the number of viable L. major parasites was evaluated in the spleen and infected lesions of mice using a limiting dilution assay. The mice were sacrificed at 8 and 12 weeks after infection; the spleens and infected lesion were aseptically removed. The spleens were homogenized in 1 ml RPMI-FCS with a sterile syringe piston. The infected lesions were transferred into the tubes containing 1 ml RPMI-FCS, and a given amount of zirconium beads. The samples were homogenized completely by bead beater for 20 s in one cycle. The homogenate was diluted with the same media in 8 serial 10-fold dilutions in each well of flat-bottom 96-well microtiter plates containing solid layer of rabbit blood agar in triplicate and kept at 25±1° C. for 7 days. The positive (presence of motile parasite) and the negative (absence of motile parasite) wells were detected using an invert microscope (CETI, UK). Data reported is the calculated mean and standard error of mean of the last positive well multiplied by a dilution factor.

Regarding statistical analysis, a one-way ANOVA statistical test was used to assess the significance of the differences among various groups. In the case of a significant F value, multiple comparison Tukey tests were used to compare the means of different treatment groups. Results with p<0.05 were considered to be statistically significant.

Cell Diffusion Studies of the in vitro penetration of the Lip-AmB formulations across mouse skin were carried out with diffusion cells, and the penetrated AmB percentage and the retained AmB percentage in the skin was determined for the formulations for up to 24 hours. The proportions of AmB in Lip-AmB-0.4%, 0.2% and 0.1% formulations that penetrated the skin were 3.49, 4.45, and 5.34 percent, respectively; and the proportions of AmB in different Lip-AmB formulations that were retained were 62.22, 54.47 and 73.92 percent for Lip-AmB-0.1, 0.2 and 0.4, respectively, as shown in TABLE 3 hereinbelow. There were no significant differences in the percentage of AmB that penetrated the skin; however, the percentage of AmB that was retained in the skin for Lip-AmB-0.4 was significantly more than Lip-AmB-0.2 and Lip-AmB-0.1. The results indicate that only around 4 percent of applied Lip-AmB formulations penetrated the skin; therefore, the systemic adverse effect of AmB would be minimal. On the other hand, the proportions of AmB in different Lip-AmB formulations that were retained in the skin were approximately 60 percent showing that most of the applied AmB remains in the different layers of skin, which would be optimal for its antileishmanial effects.

TABLE 3
Percent of penetration and retention of AmB from
liposomal AmB formulations across skin after 24 h

| Formulation | % Penetration | % Retention |
|---|---|---|
| Lip-AmB 0.1% | 5.34 ± 0.052 | 62.22 ± 0.02 |
| Lip-AmB 0.2% | 4.45 ± 0.122 | 54.47 ± 0.28 |
| Lip-AmB 0.4% | 3.49 ± 0.059 | 73.92 ± 0.30 |

### Example 7

### Effect of Liposomal AmB on L. major Promastigotes In Vitro

Minimum inhibitory concentration (MIC) of AmB in different Lip-AmB formulations against L. major promastigotes in culture were 0.625 μg/ml, as shown in TABLE 4 hereinbelow. Fungizone™ (Bristol-Myers Squibb Company, Princeton, N.J.) was used as positive control. The MIC for Fungizone™ was 0.5 μg/ml.

TABLE 4
Minimum inhibitory concentrations (MIC, μg/ml) of topical
liposomal AmB formulations against L. major promastigotes

| Formulations | MIC (μg/ml) |
|---|---|
| Lip-AmB 0.1% | 0.625 |
| Lip-AmB 0.2% | 0.625 |
| Lip-AmB 0.4% | 0.625 |
| Control empty liposome | Inactive |
| Fungizone ™ | 0.5 |

Effect of Liposomal AmB on L. major Amastigotes In Vitro

The 50% effective doses (ED50) of Lip-AmB 0.4%, 0.2% and 0.1% against L. major amastigotes in macrophages was 0.151 (μg/ml, lower and upper 95% limit: 0.0523-0.434), 0.151 (μg/ml, lower and upper 95% limit: 0.085-0.267), and 0.0856 (μg/ml, lower and upper 95% limit: 0.0147-0.50), respectively, as shown in TABLE 5 hereinbelow. Fungizone™ (Bristol-Myers Squibb Company, Princeton, N.J.) was used as a positive control. The ED50 for Fungizone™ was 0.063 (μg/ml, lower and upper 95% limit: 0.027-0.146).

TABLE 5
The effect of Lip-AmB formulation against L. major amastigotes

| | AmB Concentrations | Percent of infected |
|---|---|---|
| Formulations | (μg/ml) | cells ± SD (n = 3) |
| Control Cells | 0 | 97.33 ± 1.16 |
| Lip-AmB-0.4% | 10 | 0 ± 0 |
| Lip-AmB-0.4% | 5 | 0 ± 0 |
| Lip-AmB-0.4% | 2.5 | 0 ± 0 |
| Lip-AmB-0.4% | 1.25 | 0 ± 0 |
| Lip-AmB-0.4% | 0.5 | 58.7 ± 2.3 |
| Lip-AmB-0.4% | 0.25 | 77.33 ± 10.26 |
| Lip-AmB-0.4% | 0.125 | 81.33 ± 8.08 |
| Lip-AmB-0.4% | 0.0625 | 91.33 ± 4.16 |
| Lip-AmB-0.4% | 0.0313 | 89.33 ± 2.31 |
| Lip-AmB-0.4% | 0.0156 | 94.0 ± 3.46 |
| Lip-AmB-0.2% | 0.5 | 26.0 ± 8.72 |
| Lip-AmB-0.2% | 0.25 | 33.33 ± 3.06 |
| Lip-AmB-0.2% | 0.125 | 60.0 ± 9.17 |
| Lip-AmB-0.2% | 0.0625 | 69.33 ± 1.16 |
| Lip-AmB-0.2% | 0.0313 | 79.33 ± 7.02 |
| Lip-AmB-0.2% | 0.0156 | 89.33 ± 1.16 |
| Lip-AmB-0.1% | 0.5 | 10.67 ± 1.16 |
| Lip-AmB-0.1% | 0.25 | 33.33 ± 7.02 |
| Lip-AmB-0.1% | 0.125 | 51.33 ± 3.06 |
| Lip-AmB-0.1% | 0.0625 | 66.0 ± 14.0 |
| Lip-AmB-0.1% | 0.0313 | 69.33 ± 8.33 |
| Lip-AmB-0.1% | 0.0156 | 75.33 ± 8.08 |
| Fungizone ™ | 5 | 0 ± 0 |
| Fungizone ™ | 2.5 | 0.67 ± 1.16 |
| Fungizone ™ | 1.25 | 1.33 ± 1.16 |
| Fungizone ™ | 0.5 | 9.33 ± 4.16 |
| Fungizone ™ | 0.25 | 28.0 ± 8.0 |
| Fungizone ™ | 0.125 | 34.0 ± 7.21 |
| Fungizone ™ | 0.0625 | 39.33 ± 5.03 |
| Fungizone ™ | 0.0313 | 57.33 ± 6.43 |

Control cells represent macrophages infected with L. major without any treatment

Effect of Topical Liposomal AmB on the Size of Ulcer Induced in BALB/c Mice Infected with L. major

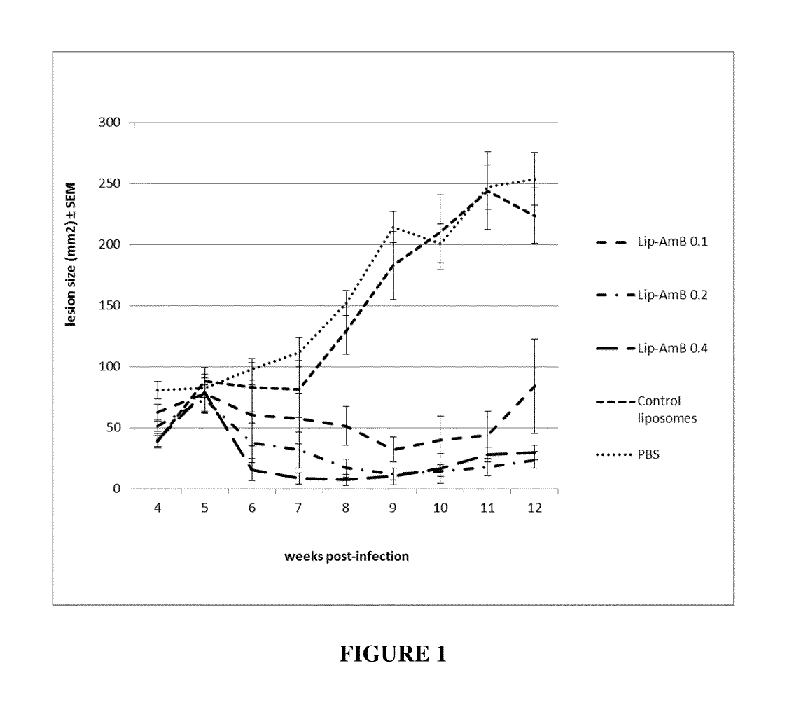

With reference now to FIG. 1 of the DRAWINGS, there is illustrated therein the effect of topical liposomal AmB on the course of disease in a BALB/c mice L. major model of cutaneous leishmaniasis. As shown, there were no significant differences (P>0.05) in lesion sizes among different groups before initiation off the treatment (FIG. 1, week 4 post-infection). Lip-AmB topically was used twice a day for 4 weeks to treat L. major lesions on BALB/c mice, and the results showed a significantly (p<0.001) smaller lesion size compared to the control groups receiving either empty liposomes or PBS. The effect of Lip-AmB 0.4% was more pronounced compared to Lip-AmB 0.2% and 0.1%.

### Effects of Topical Liposomal AmB on Splenic Parasite Burden

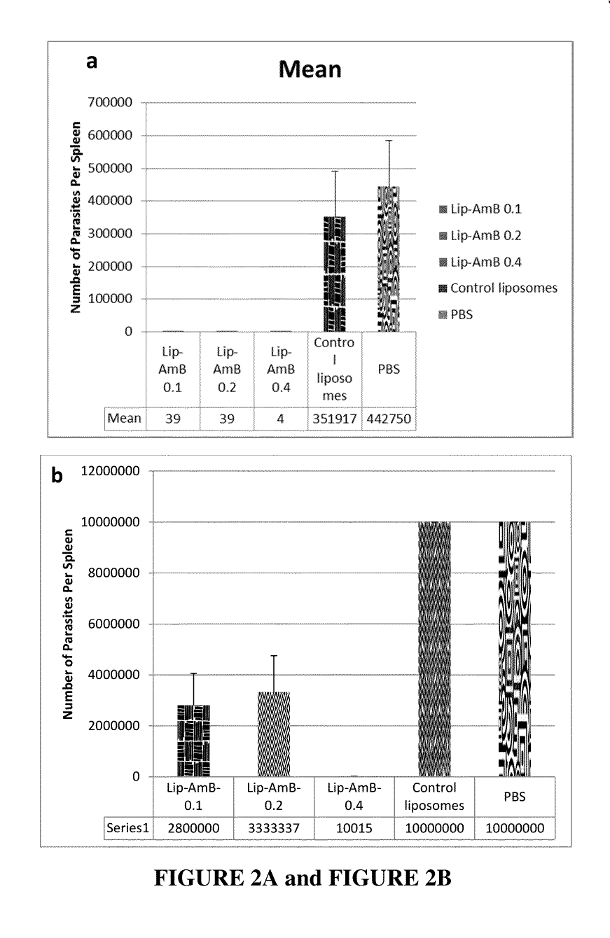

With reference now to FIGS. 2A and 2B of the DRAWINGS, at 8 weeks post-infection, the spleen parasite burden was significantly (p<0.001) lower in mice treated with Lip-AmB formulations compared to the mice that received PBS or control liposomes, as illustrated in FIG. 2A of the DRAWINGS. However, at 12 weeks post-infection, the parasite burden for Lip-AmB 0.4% was significantly (p<0.001) lower than all the other groups, as illustrated in FIG. 2B of the DRAWINGS.

### Effects of Topical Liposomal AmB on Lesion Parasite Burden

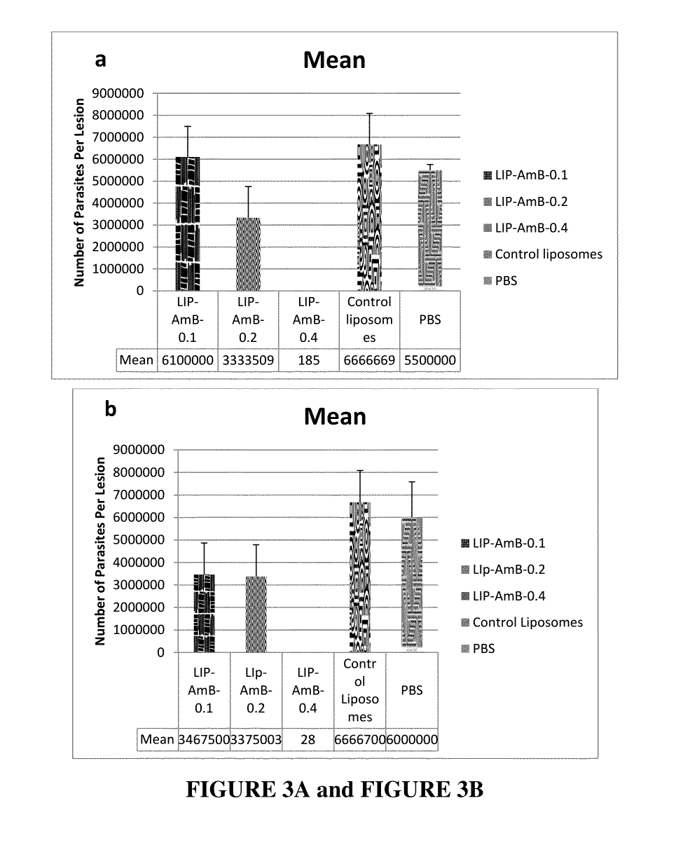

With reference now to FIGS. 3A and 3B of the DRAWINGS, the lesion parasite burden was significantly (p<0.001) lower in mice treated with Lip-AmB 0.4% compared to all the other groups, both at 8, as shown in FIG. 3A of the DRAWINGS, and at 12, as shown in FIG. 3B of the DRAWINGS, weeks post infection.

In stability studies, among different formulations, the Lip-AmB 0.4% had the best effect in the treatment of ulcers induced in BALB/c mice infected with L. major. Therefore it was decided to carry out the stability studies only with Lip-AmB 0.4% formulations.

Before starting the stability studies, three batches of Lip-AmB 0.4% formulations were prepared and characterized for their size and AmB concentration to determine whether the procedure of preparation of liposomes is reproducible. TABLE 6 hereinbelow shows the size and concentration of the Lip-AmB 0.4% in different batches. As indicated, there were no significant differences in the size and concentration of Lip-AmB 0.4% in different batches.

TABLE 6
Particle size distribution and AmB concentration of Lip-AmB-0

| | Liposome Diameter | AmB Concentration |
|---|---|---|
| Lip-AmB 0.4% | (nm) ± SD | (mg/g) ± SD |
| Batch 1 | 113.5 ± 10.4 | 4.11 ± 1.53 |
| Batch 2 | 109.7 ± 3.2 | 4.19 ± 5.69 |
| Batch 3 | 102.9 ± 3.4 | 4.23 ± 4.36 |

For stability studies, the Lip-AmB-0.4 Batch#1 was kept at 4° C. and room temperature (22° C.), and analyzed periodically for their AmB concentration, particle diameters, and biological activity of AmB, as determined by promastigote assay. The AmB concentration for Lip-AmB 0.4% was remained unchanged during storage both in 4° C. and room temperature (22° C.) up to 20 months, as shown hereinbelow in TABLES 7 and 8, respectively. When Lip-AmB 0.4% stored at 4° C. and room temperature (22° C.) there were no significant changes in the liposome size up to 20 months, as also shown hereinbelow in TABLES 9 and 10, respectively. The MIC of AmB in Lip-AmB 0.4% against L. major promastigotes in culture was 0.625 μg/ml and remained unchanged when they stored at 4° C. and room temperature up to 20 months, as shown hereinbelow in TABLE 11.

TABLE 7
AmB concentrations of liposome AmB 0.4% stored at 4° C.

| Months after liposome | AmB Concentration |
|---|---|
| preparation | (mg/g) ± SD |
| 0 | 4.11 ± 0.015 |
| 2 | 4.13 ± 0.01 |
| 4 | 4.21 ± 0.025 |
| 6 | 4.58 ± 0.04 |
| 20 | 4.00 ± 0.05 |

n = 3, Mean ± SD

TABLE 8
AmB concentrations of liposome AmB
0.4% stored at room temp (22° C.)

| Months after liposome | AmB Concentration |
|---|---|
| preparation | (mg/g) ± SD |
| 0 | 4.11 ± 0.015 |
| 1 | 4.24 ± 0.02 |
| 2 | 4.18 ± 0.05 |
| 4 | 4.14 ± 0.014 |
| 6 | 4.84 ± 0.032 |
| 20 | 4.05 ± 0.06 |

n = 3, Mean ± SD

TABLE 9
Particle size distribution of liposome AmB 0.4% stored at 4° C.

| Months after liposome | Liposome Diameter (nm) |
|---|---|
| preparation | Lip-AmB 0.4% |
| 0 | 113.5 ± 10.4 |
| 1 | 89.0 ± 20.9 |
| 3 | 90.1 ± 33.3 |
| 4 | 113.5 ± 15.1 |
| 6 | 107.2 ± 0.7 |
| 12 | 85.4 ± 10.6 |
| 20 | 91.1 ± 6.4 |

n = 3, Mean ± SD

TABLE 10
Particle size distribution of liposome
AmB 0.4% stored at room temp (22° C.)

| Months after liposome | Liposome Diameter (nm) |
|---|---|
| preparation | Lip-AmB 0.4% |
| 0 | 113.5 ± 10.4 |
| 1 | 102.5 ± 32.1 |
| 3 | 106.5 ± 9.1 |
| 4 | 115.5 ± 7.6 |
| 6 | 118.6 ± 8.7 |
| 12 | 120.8 ± 7.2 |
| 20 | 111.2 ± 0.7 |

n = 3, Mean ± SD,

TABLE 11
Minimum inhibitory concentrations (MIC, μg/ml)
of topical lip-AmB 0.4% against L. major promastigotes
stored at 4° C. and room temperature (22° C.).

| | MIC (μg/ml) | MIC (μg/ml) |
|---|---|---|
| Months after liposome | Liposome | Liposome |
| preparation | stored at 4° C. | stored at 22° C. |
| 0 | 0.625 | 0.625 |
| 1 | 0.625 | 0.625 |
| 2 | 0.625 | 0.625 |
| 4 | 0.625 | 0.625 |
| 6 | 0.625 | 0.625 |
| 12 | 0.625 | 0.625 |
| 16 | 0.625 | 0.625 |
| 17 | 0.625 | 0.625 |
| 20 | 0.625 | 0.625 |

n = 3

The aforementioned stability studies show that Lip-AmB 0.4% remains stable for more than 20 months when stored in 4° C. and room temperature, and there are no significant changes in size, concentration and biological activity of the preparation. The physical appearance of formulations also remained unchanged.

The results suggest that topical Lip-AmB 0.4% is stable in room temperature for more than 20 months, it has in vitro and in vivo antileishmanial activity, and it causes no changes in the biophysical characterization of skin in healthy volunteers. Consequently, topical Lip-AmB 0.4% is a useful tool in the treatment of cutaneous leishmaniasis, and merits further study for further enhancements not set forth herein.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the breadth or scope of the applicant's concept. Furthermore, although the present invention has been described in connection with a number of exemplary embodiments and implementations, the present invention is not so limited but rather covers various modifications and equivalent arrangements, which fall within the purview of the appended claims.

What is claimed is:

1. A topical liposomal composition for delivering hydrophobic drugs comprising:
lipid components between 5% to 15% w/w, wherein said lipid components comprise phospholipid and cholesterol;
at least one hydrophobic drug, wherein said at least one hydrophobic drug has a particle size of about 100 nm;
a solvent; and
a plurality of additives selected from the group consisting of: penetration enhancers, emulsifiers, antioxidants, buffering agents, pH adjusting agents, antimicrobial preservatives, water, and combinations thereof.

2. The topical liposomal composition according to claim 1, wherein said at least one hydrophobic drug is selected from the group consisting of cytotoxic drugs, docetaxel, paclitaxel and SN38; hormones, tamoxifen; immunosuppressive drugs, cyclosporine A; curcumin and resveratrol; corticosteroids, triamicinolone; antifungal and antileishmanial drugs, amphotricin B, miltofesine and nystatin; and combinations thereof.

3. The topical liposomal composition according to claim 1, wherein said at least one hydrophobic drug is amphotericin B (AmB).

4. The topical liposomal composition according to claim 3, wherein the amount of amphotericin B in said composition ranges from about 0.1% to about 0.5% by weight based on the total weight of the composition.

5. The topical liposomal composition according to claim 1, wherein said phospholipid is selected from a group consisting of phosphatidylcholine (PC) distearoyl phosphatidylcholine (DSPC), dipalmitoyl phosphatidylcholine (DPPC), dimirystoyitoyl phosphatidylcholine (DMPC), dilauroyl phosphatidylcholine (DLPC), soya phosphatidylcholine (SPC), egg phosphatidylcholine (EPC), hydrogenated soya phosphatidylcholine (HSPC), hydrogenated egg phosphatidylcholine (HEPC); and combinations thereof.

6. The topical liposomal composition according to claim 1, wherein liposomes in said topical liposomal composition have an average diameter less than about 150 nm.

7. The topical liposomal composition according to claim 1, wherein said plurality of additives comprise an antioxidant selected from the group consisting of vitamin E, butylated hydroxyl anisole (BHT) and mixtures thereof.

8. The topical liposomal composition according to claim 1, wherein said plurality of additives comprise a penetration enhancer selected from the group consisting of oleic acid, propylene glycol and combinations thereof.

9. The topical liposomal composition according to claim 1, wherein said plurality of additives comprise an antimicrobial preservative selected from the group consisting of methyl parabene (MP), propyl parabene (PP), phenol and combinations thereof.

10. The topical liposomal composition according to claim 1, wherein said solvent comprises at least two solvents for dissolving said lipid components, wherein said at least the two solvents are propylene glycol and glycerol in a combined amount of about 1% to about 25% by weight based on the total weight of the composition.

11. The topical liposomal composition according to claim 1, wherein said solvent comprises dimethyl sulfoxide (DMSO).

12. The topical liposomal composition according to claim 11, wherein said at least one hydrophobic drug is AmB, wherein said AmB ranges from about 1% to about 10% by weight of the total weight of the composition.

13. The topical liposomal composition according to claim 1, wherein the composition is formulated in a form selected from the group consisting of a cream, a serum, a lotion, a gel, and combinations thereof.

14. A method for preparing a topical liposomal composition comprising:
dissolving lipid components in a solvent;
mixing the dissolved lipid components in said solvent with a plurality of additives selected from the group consisting of a penetration enhancer, an antimicrobial preservative, an antioxidant, and combinations thereof, forming a lipid phase thereof:
melting said lipid phase, forming a uniform dissolved lipid phase;
dissolving and mixing at least one hydrophobic drug into the solvent in the uniform lipid phase, forming an admixture thereof;
heating said admixture;
dissolving an emulsifier into a solution, preparing an aqueous phase thereof;
heating said solution in said aqueous phase; and
mixing and homogenizing the admixture and said solution in said aqueous phase to obtain said topical liposomal composition.

15. The method according to claim 14, wherein said solution comprises triethanolamine in water or triethanolamine in a buffer solution.

16. The method according to claim 14, wherein said at least one hydrophobic drug is selected from the group consisting of cytotoxic drugs, docetaxel, paclitaxel and SN38; hormones, tamoxifen; immunosuppressive drugs, cyclosporine A; curcumin and resveratrol; corticosteroids, triamicinolone; antifungal and antileishmanial drugs, amphotricin B, miltofesine and nystatin; and combinations thereof.

17. The method according to claim 14, wherein said at least one hydrophobic drug is Amphotericin B.

18. The method according to claim 14, wherein in said heating said lipid phase and aqueous phase, the temperature is between about 60-75 degrees C.

19. The method according to claim 14, wherein said solvent comprises at least two solvents for dissolving said lipid components, wherein said at least two solvents are propylene glycol and glycerol in a combined amount of about 1% to about 25% by weight based on the total weight of the composition.

20. The method according to claim 14, wherein said solvent comprises dimethyl sulfoxide (DMSO).

21. The method according to claim 20, wherein the amount of said dimethyl sulfoxide (DMSO) ranges from about 1% to about 10% by weight of the total weight of the composition.

22. The method according to claim 14, wherein liposomes in said topical liposomal composition have an average diameter less than about 150 nm

23. The method according to claim 14, wherein said lipid components comprise phospholipid and cholesterol.

24. The method according to claim 23, wherein said phospholipid is selected from the group consisting of phosphatidylcholine (PC), distearoyl phosphatidylcholine (DSPC), dipalmitoyl phosphatidylcholine (DPPC), dimirystoyitoyl phosphatidylcholine (DMPC), dilauroyl phosphatidylcholine (DLPC), soya phosphatidylcholine (SPC), egg phosphatidylcholine (EPC), hydrogenated soya phosphatidylcholine (HSPC), hydrogenated egg phosphatidylcholine (HEPC) and combinations thereof; and
wherein the amount of said lipid component ranges between about 5% to about 20% w/w.

25. The method according to claim 14, wherein said plurality of additives comprise an antioxidant selected from the group consisting of vitamin E and butylated hydroxyl anisole (BHT) and mixtures thereof.

26. The method according to claim 14, wherein said plurality of additives comprise a penetration enhancer selected from the group consisting of oleic acid, propylene glycol and combinations thereof.

27. The method according to claim 14, wherein said plurality of additives comprise an antimicrobial preservative selected from the group consisting of methyl parabene (MP), propyl parabene (PP), phenol and combinations thereof.
